(19) United States
(12) Patent Application Publication
(10) Pub. No.: US 2010/0130861 A1
(43) Pub. Date: May 27, 2010

(54) ULTRASONIC DIAGNOSTIC APPARATUS AND PROGRAM

(76) Inventor: Tadashi Shimazaki, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/625,138

(22) Filed: Nov. 24, 2009

(30) Foreign Application Priority Data
Nov. 26, 2008 (JP) 2008-300872

(51) Int. Cl. A61B 8/14

(52) U.S. Cl. 600/443

(57) ABSTRACT

An ultrasonic diagnostic apparatus includes a transmit-receive unit for driving an ultrasonic probe to perform transmission/reception of ultrasound and outputting each echo signal received by the ultrasonic probe, wherein transmit-receive unit causes a first elastic image ultrasonic transmission/reception operation and a second elastic image ultrasonic transmission/reception operation to be performed at predetermined time intervals for obtaining echo signals each used to generate an elastic image of the biological tissue, and causes non-elastic image ultrasonic transmission/reception to be performed for obtaining an echo signal used to generate a non-elastic image related to the biological tissue. An elastic image processing unit calculates a physical quantity related to elasticity of the biological tissue based on the two echo signals generates the elastic image based on the physical quantity, and a non-elastic image processing unit generates the non-elastic image.

### CROSS REFERENCE TO RELATED APPLICATIONS

This application claims the benefit of Japanese Patent Application No. 2008-300872 filed Nov. 26, 2008, which is hereby incorporated by reference in its entirety.

### BACKGROUND OF THE INVENTION

The embodiments described herein relate to an ultrasonic diagnostic apparatus, and particularly to an ultrasonic diagnostic apparatus and a program capable of representing an elastic image indicative of the hardness or softness of a biological tissue.

An ultrasonic diagnostic apparatus wherein an elastic image indicative of the hardness or softness of a biological tissue is displayed on a normal B-mode image in overlaid form, has been disclosed in, for example, a Japanese Unexamined Patent Publication No. 2000-60853 or the like. In this type of ultrasonic diagnostic apparatus, the elastic image is formed as follows: The transmission/reception of ultrasound is first carried out while repeating pressure by an ultrasonic probe and its relaxation in a state in which the ultrasonic probe is being brought into contact with a subject. Then, a physical quantity related to elasticity of each part in the biological tissue is calculated from two echo signals different in time from each other, which have been obtained by performing such transmission/reception of ultrasound. The elasticity of the biological tissue is imaged in color based on the calculated physical quantity related to the elasticity.

When the elastic image obtained in the above-described manner is displayed, it has been practised to display an elastic image on a B-mode image indicative of the form of a biological tissue in overlaid form with a view toward making it possible to grasp which part of biological tissue represents an elastic image. In this case, there is a need to form the elastic image and the B-mode image from the corresponding echo signal.

Here, there is, for example, a case in which a condition for transmitting ultrasound for obtaining an echo signal used to generate an elastic image and a condition for transmitting ultrasound for obtaining an echo signal used to generate a B-mode image differ from each other. In such a case, there is a need to perform in different time phases, the transmission/reception of the ultrasound for obtaining the echo signal used to generate the elastic image and the transmission/reception of the ultrasound for obtaining the echo signal used to generate the B-mode image. Since the two echo signals different in time from each other are required to generate the elastic image as described above, the transmission/reception of ultrasound for obtaining an echo signal used to generate an elastic image has heretofore been carried out twice and thereafter the transmission/reception of ultrasound for obtaining an echo signal used to generate a B-mode image has been carried out.

Meanwhile, there is a need to calculate the physical quantity related to the elasticity of the biological tissue from the two echo signals different in time acquired while repeating the pressure to the biological tissue and its relaxation as described above with a view toward generating the elastic image. Therefore, the physical quantity cannot be calculated properly so long as sufficient application of pressure or reduction in pressure is not done while the two echo signals are acquired, so that a satisfactory elastic image cannot be obtained. It is thus necessary to perform the double ultrasonic transmission/reception for obtaining the echo signal used to generate the elastic image at a certain degree of intervals. Therefore, after the double ultrasonic transmission/reception for obtaining the echo signal used to generate the elastic image has been performed at the certain degree of intervals, the ultrasonic transmission/reception for obtaining the echo signal used to generate the B-mode image is carried out, thus encountering a difficulty in terms of a frame rate.

It is desirable that the problems described previously are solved.

### BRIEF DESCRIPTION OF THE INVENTION

The invention according to a first aspect provides an ultrasonic diagnostic apparatus including an ultrasonic probe for performing transmission/reception of ultrasound on a biological tissue; a transmit-receive unit for driving the ultrasonic probe to perform the transmission/reception of the ultrasound and outputting each echo signal received by the ultrasonic probe, the transmit-receive unit causing one elastic image ultrasonic transmission/reception and another elastic image ultrasonic transmission/reception for obtaining echo signals each used to generate an elastic image of the biological tissue to be performed at predetermined time intervals and causing non-elastic image ultrasonic transmission/reception for obtaining an echo signal used to generate a non-elastic image related to the biological tissue, other than the elastic image to be performed between the one elastic image ultrasonic transmission/reception and said another elastic image ultrasonic transmission/reception; an elastic image processing unit for calculating a physical quantity related to elasticity of the biological tissue, based on the two echo signals different in time obtained by the one elastic image ultrasonic transmission/reception and said another elastic image ultrasonic transmission/reception and generating the elastic image, based on the physical quantity; and a non-elastic image processing unit for generating the non-elastic image, based on the echo signal obtained by the non-elastic image ultrasonic transmission/reception.

The invention according to a second aspect provides an ultrasonic diagnostic apparatus wherein in the invention according to the first aspect, the transmit-receive unit outputs the echo signal to the elastic image processing unit upon the elastic image ultrasonic transmission/reception, whereas upon the non-elastic image ultrasonic transmission/reception, the transmit-receive unit outputs the echo signal to the non-elastic image processing unit.

The invention according to a third aspect provides an ultrasonic diagnostic apparatus wherein in the invention according to the first aspect, the non-elastic image processing unit is a B-mode image processing unit for generating a B-mode image, and the transmit-receive unit causes B-mode image ultrasonic transmission/reception for obtaining an echo signal used to generate a B-mode image to be performed as the non-elastic image ultrasonic transmission/reception.

The invention according to a fourth aspect provides an ultrasonic diagnostic apparatus wherein in the invention according to the third aspect, the transmit-receive unit outputs the echo signal to the elastic image processing unit upon the elastic image ultrasonic transmission/reception, whereas upon the B-mode image ultrasonic transmission/reception, the transmit-receive unit outputs the echo signal to the B-mode image processing unit.

The invention according to a fifth aspect provides an ultrasonic diagnostic apparatus including a combination unit for combining the elastic image and the B-mode image together in the invention according to each of the third and fourth aspects.

The invention according to a sixth aspect provides an ultrasonic diagnostic apparatus wherein in the invention according to the first aspect, the non-elastic image processing unit is a color Doppler image processing unit for generating a color Doppler image, and the transmit-receive unit causes color Doppler image ultrasonic transmission/reception for obtaining an echo signal used to generate a color Doppler image to be performed as the non-elastic image ultrasonic transmission/reception.

The invention according to a seventh aspect provides an ultrasonic diagnostic apparatus wherein in the invention according to the sixth aspect, the transmit-receive unit outputs the echo signal to the elastic image processing unit upon the elastic image ultrasonic transmission/reception, whereas upon the color Doppler image ultrasonic transmission/reception, the transmit-receive unit outputs the echo signal to the color Doppler image processing unit.

The invention according to an eighth aspect provides an ultrasonic diagnostic apparatus wherein in the invention according to each of the sixth and seventh aspects, the color Doppler image processing unit has an extraction part for extracting a signal indicative of a blood flow component from the echo signal.

The invention according to a ninth aspect provides an ultrasonic diagnostic apparatus including a combination unit for combining the elastic image and the color Doppler image together in the invention according to any one of the sixth through eight aspects.

The invention according to a tenth aspect provides an ultrasonic diagnostic apparatus wherein in the invention according to the first aspect, the non-elastic image processing unit is provided in plural form and the respective non-elastic image processing units generate non-elastic images different in type from one another, and the transmit-receive unit causes transmission/reception for the respective non-elastic images to be performed as the non-elastic image ultrasonic transmission/reception.

The invention according to an eleventh aspect provides an ultrasonic diagnostic apparatus wherein in the invention according to the tenth aspect, the transmit-receive unit outputs the echo signal to the elastic image processing unit upon the elastic image ultrasonic transmission/reception, whereas upon the non-elastic image ultrasonic transmission/reception, the transmit-receive unit outputs the echo signal to its corresponding non-elastic image processing unit of the non-elastic image processing units.

The invention according to a twelfth aspect provides an ultrasonic diagnostic apparatus wherein in the invention according to the tenth aspect, each of the non-elastic image processing units is a B-mode image processing unit for generating a B-mode image and a color Doppler image processing unit for generating a color Doppler image, and the transmit-receive unit causes B-mode image ultrasonic transmission/reception for obtaining an echo signal used to generate the B-mode image and color Doppler image ultrasonic transmission/reception for obtaining an echo signal used to generate the color Doppler image to be performed as the non-elastic image ultrasonic transmission/reception.

The invention according to a thirteenth aspect provides an ultrasonic diagnostic apparatus wherein in the invention according to the twelfth aspect, the transmit-receive unit outputs the echo signal to the elastic image processing unit upon the elastic image ultrasonic transmission/reception, the transmit-receive unit outputs the echo signal to the B-mode image processing unit upon the B-mode image ultrasonic transmission/reception, and the transmit-receive unit outputs the echo signal to the color Doppler image processing unit upon the color Doppler image ultrasonic transmission/reception.

The invention according to a fourteenth aspect provides an ultrasonic diagnostic apparatus including a combination unit for combining the B-mode image and the color Doppler image together in the invention according to each of the twelfth and thirteenth aspects.

The invention according to a fifteenth aspect provides a program for causing a computer of an ultrasonic diagnostic apparatus to execute a transmitting/receiving function for driving an ultrasonic probe to cause transmission/reception of ultrasound to be performed on a biological tissue and outputting echo signals received by the ultrasonic probe, said transmitting/receiving function causing one elastic image ultrasonic transmission/reception and another elastic image ultrasonic transmission/reception for obtaining echo signals each used to generate an elastic image of the biological tissue to be performed at predetermined time intervals and causing non-elastic image ultrasonic transmission/reception for obtaining an echo signal used to generate a non-elastic image related to the biological tissue, other than the elastic image to be performed between the one elastic image ultrasonic transmission/reception and said another elastic image ultrasonic transmission/reception; an elastic image generating function for calculating a physical quantity related to elasticity of the biological tissue, based on the two echo signals different in time obtained by the one elastic image ultrasonic transmission/reception and said another elastic image ultrasonic transmission/reception and generating the elastic image, based on the physical quantity; and a non-elastic image generating function for generating the non-elastic image, based on the echo signal obtained by the non-elastic image ultrasonic transmission/reception.

In embodiments, since the non-elastic image ultrasonic transmission/reception is carried out between the one elastic image ultrasonic transmission/reception and another elastic image ultrasonic transmission/reception at the predetermined time intervals, a frame rate can be enhanced.

Further objects and advantages of the embodiments described herein will be apparent from the following description of the preferred embodiments of the invention as illustrated in the accompanying drawings.

### BRIEF DESCRIPTION OF THE DRAWINGS

FIG. 1 is a block diagram showing a configuration of an ultrasonic diagnostic apparatus according to a first embodiment of the invention.

FIG. 2 is a block diagram showing a configuration of a B-mode image processing unit employed in the ultrasonic diagnostic apparatus shown in FIG. 1.

FIG. 3 is a block diagram showing a configuration of an elastic image processing unit employed in the ultrasonic diagnostic apparatus shown in FIG. 1.

FIG. 4 is a diagram showing timings provided to perform ultrasonic transmission/reception for elastic images and ultrasonic transmission/reception for B-mode images in the ultrasonic diagnostic apparatus shown in FIG. 1.

FIG. 5 is a block diagram showing a configuration of an ultrasonic diagnostic apparatus according to a second embodiment of the invention.

FIG. 6 is a block diagram showing a detailed configuration of a color Doppler image processing unit employed in the ultrasonic diagnostic apparatus shown in FIG. 5.

FIG. 7 is a diagram showing timings provided to perform ultrasonic transmission/reception for elastic images, ultrasonic transmission/reception for B-mode images and ultrasonic transmission/reception for color Doppler images in the ultrasonic diagnostic apparatus shown in FIG. 5.

### DETAILED DESCRIPTION OF THE INVENTION

An ultrasonic diagnostic apparatus and a program according to the invention will hereinafter be described in detail based on the accompanying drawings.

### First Embodiment

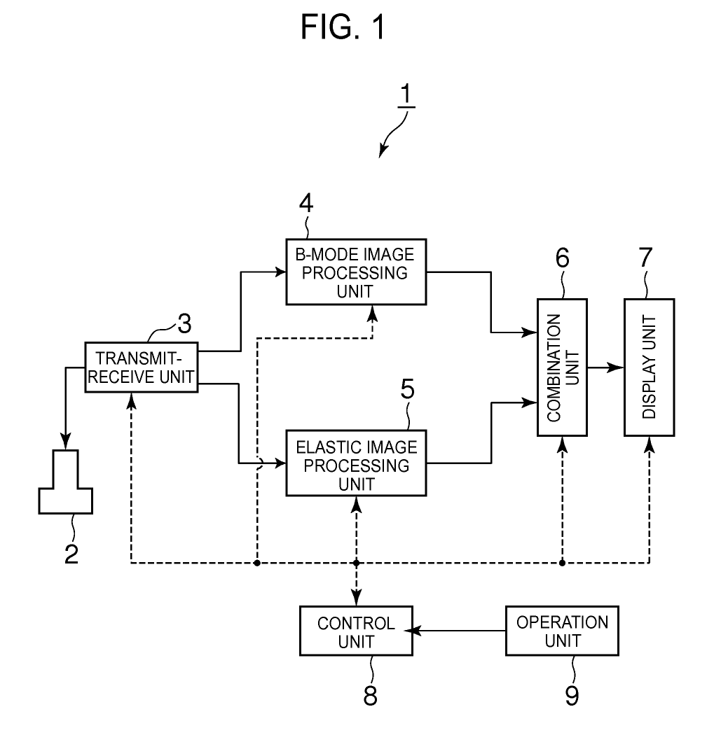

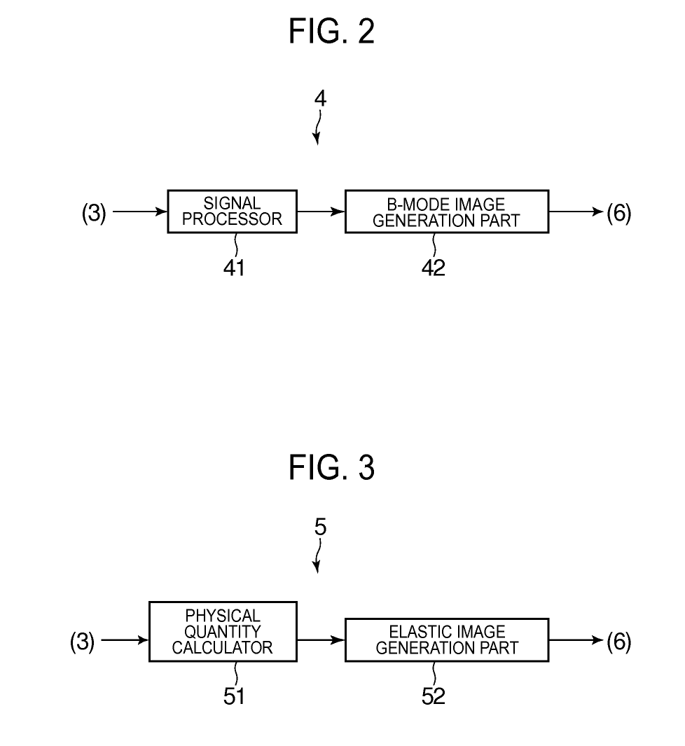

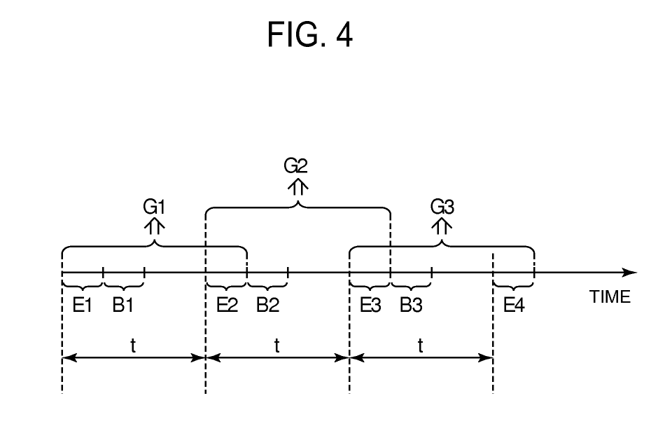

A first embodiment will first be explained. FIG. 1 is a block diagram showing a configuration of an ultrasonic diagnostic apparatus according to the first embodiment of the invention, FIG. 2 is a block diagram showing a configuration of a B-mode image processing unit employed in the ultrasonic diagnostic apparatus shown in FIG. 1, FIG. 3 is a block diagram showing a configuration of an elastic image processing unit employed in the ultrasonic diagnostic apparatus shown in FIG. 1, and FIG. 4 is a diagram showing timings provided to perform ultrasonic transmission/reception for elastic images and ultrasonic transmission/reception for B-mode images in the ultrasonic diagnostic apparatus shown in FIG. 1, respectively.

The ultrasonic diagnostic apparatus 1 shown in FIG. 1 is equipped with an ultrasonic probe 2, a transmit-receive unit 3, a B-mode image processing unit 4, an elastic image processing unit 5, a combination unit 6 and a display unit 7. Further, the ultrasonic diagnostic apparatus 1 includes a controller or control unit 8 and an operation unit 9.

The ultrasonic probe 2 performs the transmission/reception of ultrasound on a subject. The ultrasonic probe 2 performs the transmission/reception of the ultrasound while repeating pressure and relaxation in a state of being brought into contact with the subject thereby to make it possible to obtain elastic images.

The transmit-receive unit 3 drives the ultrasonic probe 2 to perform the transmission/reception of the ultrasound. The transmit-receive unit 3 performs signal processing such as phasing-adding processing on each echo signal obtained by the transmission/reception of the ultrasound. Then, the transmit-receive unit 3 outputs the echo signals subjected to the signal processing to the B-mode image processing unit 4 and the elastic image processing unit 5. The details of the transmission/reception of the ultrasound by the transmit-receive unit 3 will be described later. The transmit-receive unit 3 is one example illustrative of an embodiment of a transmit-receive unit according to the invention.

The B-mode image processing unit 4 has a signal processor 41 and a B-mode image generating part 42. The signal processor 41 performs signal processing such as logarithmic compression, envelop detection or the like on the echo signals set every sound ray from the transmit-receive unit 3. The B-mode image generation part 42 generates B-mode image frame data, based on a signal outputted from the signal processor 41. The B-mode image processing unit 4 is one example illustrative of an embodiment of a non-elastic image processing unit according to the invention.

The elastic image processing unit 5 has a physical quantity calculator 51 and an elastic image generating part 52. The elastic image processing unit 5 is one example illustrative of an embodiment of an elastic image processing unit according to the invention. Described concretely, the physical quantity calculator 51 calculates displacements produced due to deformation of respective parts or regions of a biological tissue as physical quantities related to the elasticity of the biological tissue, based on the echo signals set every sound ray from the transmit-receive unit 3. Described specifically, the physical quantity calculator 51 performs an autocorrelation calculation on two echo signals different in time from each other on the same sound ray thereby to calculate displacements of the respective parts.

The elastic image generation part 52 generates elastic image frame data, based on the displacements due to the deformation of the biological tissue, which have been calculated by the physical quantity calculator 51. Described concretely, the elastic image generation part 52 provides hue information about the red, green and blue according to the displacements and generates elastic image frame data having color information.

The combination unit 6 has a frame memory (not shown) that stores therein the B-mode image frame data generated by the B-mode image processing unit 4 and the elastic image frame data generated by the elastic image processing unit 5. The combination unit 6 combines these B-mode image frame data and elastic image frame data together. The combination unit 6 is one example illustrative of an embodiment of a combination unit according to the invention. An ultrasonic image obtained by combining them using the combination unit 6 is displayed on the display unit 7.

The control unit 8 has a CPU (Central Processing Unit) or the like. The control unit 8 controls the transmit-receive unit 3, the B-mode image processing unit 4, the elastic image processing unit 5, the combination unit 6 and the display unit 7, based on an operation input signal inputted from the operation unit 9 and a program stored in advance.

The operation of the ultrasonic diagnostic apparatus 1 according to the present embodiment will now be explained. The transmit-receive unit 3 drives the ultrasonic probe 2 to perform the transmission of ultrasound to a biological tissue of the subject, thereby acquiring echo signals. Upon the transmission/reception of the ultrasound from the ultrasonic probe 2, the pressure applied by the ultrasonic probe 2 and its relaxation are repeated in a state in which the ultrasonic probe 2 is being brought into contact with the surface of the biological tissue.

The transmission/reception of the ultrasound will be described in detail. As shown in FIG. 4, the transmit-receive unit 3 causes ultrasonic transmission/reception E1, E2, E3, E4, . . . for elastic images to be executed at intervals of time t. Here, the time t corresponds to the time from the start of the elastic image ultrasonic transmission/reception to the start of the following elastic image ultrasonic transmission/reception. The time t is set to such time that the next elastic image ultrasonic transmission/reception is started after the completion of the elastic image ultrasonic transmission/reception with an interval left therebetween.

Incidentally, as will be described later, the calculation of each displacement by the physical quantity calculator 51 is performed based on two echo signals obtained by the elastic image ultrasonic transmission/reception adjacent to each other on a time sequence basis to generate an elastic image. Based on the echo signal obtained by the elastic image ultrasonic transmission/reception E1 and the echo signal obtained by the elastic image ultrasonic transmission/reception E2, for example, the calculation of each displacement by the physical quantity calculator 51 is performed to generate elastic image frame data EFD1. The calculation of each displacement by the physical quantity calculator 51 is performed based on the echo signal obtained by the elastic image ultrasonic transmission/reception E2 and an echo signal obtained by the elastic image ultrasonic transmission/reception E3 to generate an elastic image frame data EFD2 corresponding to the following frame. Further, the calculation of each displacement by the physical quantity calculator 51 is performed based on the echo signal obtained by the elastic image ultrasonic transmission/reception E3 and an echo signal obtained by the elastic image ultrasonic transmission/reception E4 to generate elastic image frame data EFD3 corresponding to the next frame.

Here, one elastic image ultrasonic transmission/reception and another elastic image ultrasonic transmission/reception correspond to their corresponding ultrasonic transmission/reception for elastic images, adjacent to each other on a time sequence basis. For example, one elastic image ultrasonic transmission/reception and another elastic image ultrasonic transmission/reception where the elastic image frame data EFD1 is generated respectively correspond to the elastic image ultrasonic transmission/reception E1 and the elastic image ultrasonic transmission/reception E2. One elastic image ultrasonic transmission/reception and another elastic image ultrasonic transmission/reception where the elastic image frame data EFD2 is generated respectively correspond to the elastic image ultrasonic transmission/reception E2 and the elastic image ultrasonic transmission/reception E4. Further, one elastic image ultrasonic transmission/reception and another elastic image ultrasonic transmission/reception respectively correspond to the ultrasonic transmission/reception E3 for the elastic image and the ultrasonic transmission/reception E4 for the elastic image.

The transmit-receive unit 3 performs ultrasonic transmission/reception B1, B2, B3, . . . for B-mode images, for obtaining echo signals used to produce the B-mode images at the B-mode image processing unit 4 between the one elastic image ultrasonic transmission/reception and another elastic image ultrasonic transmission/reception. For example, the transmit-receive unit 3 causes the ultrasonic transmission/reception B1 for the B-mode image to be performed between the elastic image ultrasonic transmission/reception E1 and the elastic image ultrasonic transmission/reception E2. The transmit-receive unit 3 causes the ultrasonic transmission/reception B2 for the B-mode image to be performed between the elastic image ultrasonic transmission/reception E2 and the elastic image ultrasonic transmission/reception E3. Further, the transmit-receive unit 3 causes the ultrasonic transmission/reception B3 for the B-mode image to be performed between the elastic image ultrasonic transmission/reception E3 and the elastic image ultrasonic transmission/reception E4.

When the respective elastic image ultrasonic transmission/reception E1, E2, E3, E4, . . . are being carried out, the transmit-receive unit 3 outputs echo signals to the elastic image processing unit 5. When the respective B-mode image ultrasonic transmission/reception B1, B2, B3, . . . are being carried out, the transmit-receive unit 3 outputs echo signals to the B-mode image processing unit 4.

The B-mode image processing unit 4 sequentially generates B-mode image frame data, based on the echo signals from the transmit-receive unit 3. For example, the B-mode image processing unit 4 generates B-mode image frame data BFD1, based on the echo signal obtained by the B-mode image ultrasonic transmission/reception B1 and generates B-mode image frame data BFD2, based on the echo signal obtained by the B-mode image ultrasonic transmission/reception B2. Further, the B-mode image processing unit 4 generates B-mode image frame data BFD3, based on the echo signal obtained by the B-mode image ultrasonic transmission/reception B3. The respective B-mode image frame data BFD1, BFD2, BFD3, . . . are outputted to the combination unit 6 and sequentially stored in the frame memory (not shown).

The elastic image processing unit 5 sequentially generates elastic image frame data, based on the two echo signals different in time from the transmit-receive unit 3. For example, the elastic image processing unit 5 performs the calculation of a displacement by the physical quantity calculator 51, based on the echo signal obtained by the ultrasonic transmission/reception E1 for the elastic image and the echo signal obtained by the ultrasonic transmission/reception E2 for the elastic image. Hence, the elastic image generation part 52 generates elastic image frame data EFD1 from the result of calculation. Similarly, the elastic image processing unit 5 generates elastic image frame data EFD2, based on the echo signal obtained by the ultrasonic transmission/reception E2 for the elastic image and the echo signal obtained by the ultrasonic transmission/reception E3 for the elastic image. The elastic image processing unit 5 generates elastic image frame data EFD3, based on the echo signal obtained by the ultrasonic transmission/reception E3 for the elastic image and the echo signal obtained by the ultrasonic transmission/reception E4 for the elastic image. The respective elastic image frame data EFD1, EFD2, EFD3, . . . are outputted to the combination unit 6 and sequentially stored in the frame memory (not shown).

The combination unit 6 combines both B-mode image frame data and elastic image frame data together to generate an ultrasonic or ultrasonic image obtained by overlaying each elastic image on its corresponding B-mode image. The ultrasonic image is outputted to the display unit 7 and displayed thereon. Described concretely, the combination unit 6 combines B-mode image frame data BFD1 and elastic image frame data EFD1 together to generate an ultrasonic image G1, which in turn is displayed on the display unit 7. The combination unit 6 generates an ultrasonic image G2 from B-mode image frame data BFD2 and elastic image frame data EFD2 and generates an ultrasonic image G3 from B-mode image frame data BFD3 and elastic image frame data EFD3. These ultrasonic images G2 and G3 are sequentially displayed on the display unit 7.

According to the ultrasonic diagnostic apparatus 1 according to the present embodiment as described above, for example, the ultrasonic image G1 is generated based on the echo signals obtained by the ultrasonic transmission/reception E1 and E2 for the elastic images and the echo signal obtained by the ultrasonic transmission/reception B1 for the B-mode image. The ultrasonic image G2 is generated based on the echo signals obtained by the ultrasonic transmission/reception E2 and E3 for the elastic images and the echo signal obtained by the ultrasonic transmission/reception B2 for the B-mode image. Further, the ultrasonic image G3 is generated based on the echo signals obtained by the ultrasonic transmission/reception E3 and E4 for the elastic images and the echo signal obtained by the ultrasonic transmission/reception B3 for the B-mode image. That is, in the ultrasonic diagnostic apparatus 1 according to the present embodiment, the B-mode image ultrasonic transmission/reception is carried out between one elastic image ultrasonic transmission/reception and another elastic image ultrasonic transmission/reception both used to generate one ultrasonic image. Here, in the conventional example, one elastic image ultrasonic transmission/reception and another elastic image ultrasonic transmission/reception both used to generate one ultrasonic image are carried out at predetermined intervals in a manner similar to the present embodiment, and the B-mode image ultrasonic transmission/reception is thereafter performed. Thus, according to the present embodiment, a frame rate can be enhanced as compared with such a conventional example.

According to the present embodiment as well, the displacement in position between the B-mode image and the elastic image with respect to the ultrasonic image can be suppressed. This will be explained citing as an example the case where the ultrasonic image G1 is generated, for example. When the autocorrelation calculation is performed between the echo signal obtained by the ultrasonic transmission/reception E1 for the elastic image and the echo signal obtained by the ultrasonic transmission/reception E2 for the elastic image on the basis of the echo signal obtained by the elastic image ultrasonic transmission/reception E1 thereby to generate the elastic image frame data EFD1, the time interval between the ultrasonic transmission/reception B1 for the B-mode image and the ultrasonic transmission/reception E1 for the elastic image becomes shorter than conventional. Since the biological tissue is moving due to the pressure and relaxation by the ultrasonic probe 2 upon the transmission/reception of ultrasound here, the time interval between the B-mode image ultrasonic transmission/reception B1 and the elastic image ultrasonic transmission/reception E1 becomes short, so that the displacement in position between the B-mode image and the elastic image can be suppressed.

### Second Embodiment

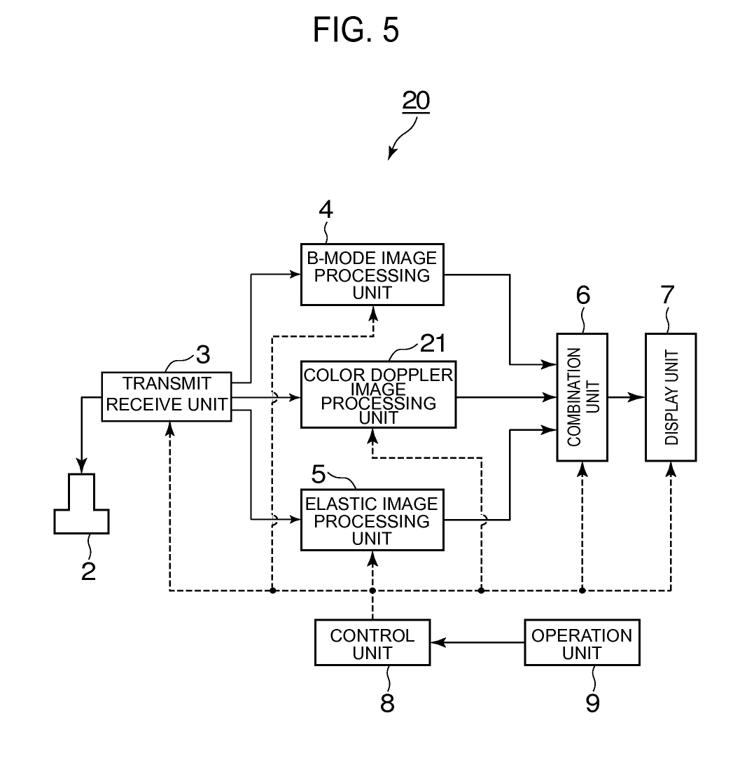

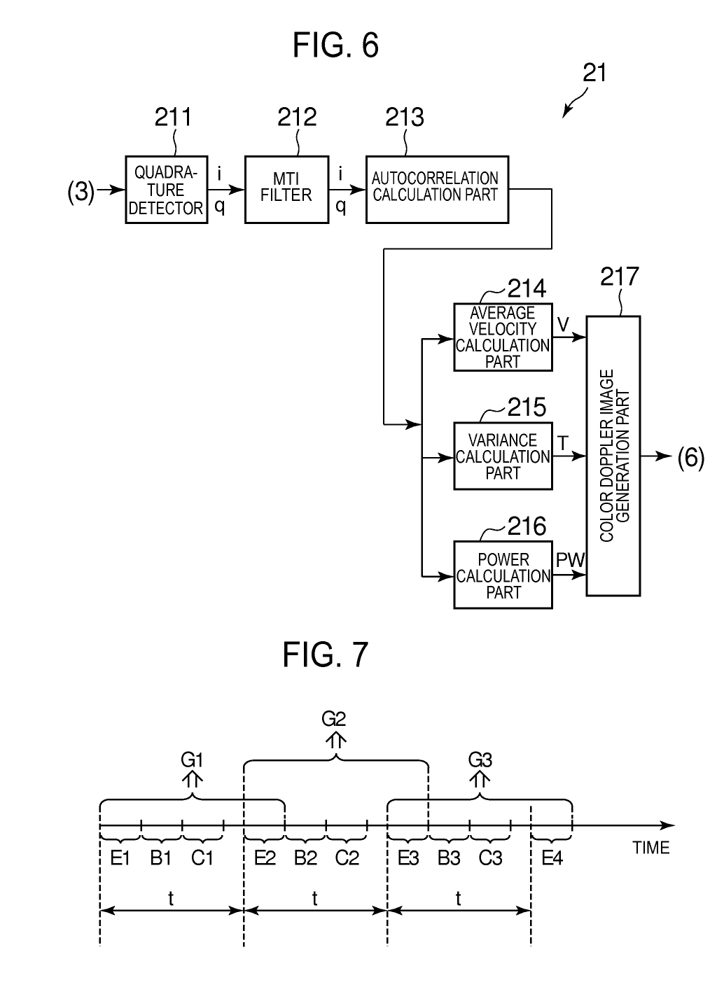

A second embodiment will next be explained. FIG. 5 is a block diagram showing a configuration of an ultrasonic diagnostic apparatus according to the second embodiment of the invention, FIG. 6 is a block diagram showing a detailed configuration of a color Doppler image processing unit employed in the ultrasonic diagnostic apparatus shown in FIG. 5, and FIG. 7 is a diagram showing timings provided to perform ultrasonic transmission/reception for elastic images, ultrasonic transmission/reception for B-mode images and ultrasonic transmission/reception for color Doppler images in the ultrasonic diagnostic apparatus shown in FIG. 5, respectively. In FIG. 5, the same reference numerals are respectively attached to the same components as those in the first embodiment, and their detailed explanations will therefore be omitted.

The ultrasonic diagnostic apparatus 20 shown in FIG. 5 is equipped with a color Doppler image processing unit 21 for generating color Doppler image frame data, based on echo signals outputted from the transmit-receive unit 3. The present color Doppler image processing unit 21 is one example illustrative of an embodiment of a non-elastic image processing unit in the invention.

As shown in FIG. 6, the color Doppler image processing unit 21 has a quadrature detector 211, an MTI (Moving Target Indication) filter 212, an autocorrelation calculation part 213, an average flow rate or velocity calculation part 214, a variance calculation part 215, a power calculation part 216 and a color Doppler image generation part 217.

The color Doppler image processing unit 21 first quadrature-detects an echo signal obtained by performing ultrasonic transmission/reception for a color Doppler image by the transmit-receive unit 3, through the quadrature detector 211. Next, the MTI filter 212 MTI-processes the post-quadrature detection signal thereby to remove a component caused by the motion of a biological tissue, which occurs due to the pressure and relaxation of the biological tissue by the ultrasonic probe 2, thus resulting in the extraction of only a signal indicative of a blood flow component. Here, the MTI filter 212 is one example illustrative of an embodiment of an extraction part in the invention.

The autocorrelation calculation part 213 performs an autocorrelation calculation on a signal outputted from the MTI filter 212. The average velocity calculation part 214 determines a flow velocity V in response to the output of the autocorrelation calculation part 213. The variance calculation part 215 determines a variance T of a flow velocity in response to the output of the autocorrelation calculation part 213. The power calculation part 216 determines power PW in response to the output of the autocorrelation calculation part 213.

The color Doppler image generation part 217 generates color Doppler image frame data, based on the flow velocity V, variance T and power PW. As the color Doppler image frame data, at least one of flow velocity distribution image frame data obtained by combining the flow velocity V and variance T together, power Doppler image frame data using the power PW or variance-added power Doppler image frame data obtained by combining the power PW and variance T together, and variance image frame data using a variance T is generated. The color Doppler image frame data is outputted to the combination unit 6.

Incidentally, the color Doppler image frame data has such hue information that it can be distinguished from each elastic image when disposed on the display unit 7 as a color Doppler image.

The operation of the ultrasonic diagnostic apparatus 20 according to the present embodiment will now be described. In the present embodiment, color Doppler image frame data outputted from the color Doppler image processing unit 21 is stored in the corresponding frame memory (not shown) of the combination unit 6 in addition to B-mode image frame data outputted from the B-mode image processing unit 4 and elastic image frame data outputted from the elastic image processing unit 5. The combination unit 6 combines the B-mode image frame data, the color Doppler image frame data and the elastic image frame data together to generate an ultrasonic image with both a color Doppler image and an elastic image overlaid on a B-mode image.

The transmission/reception of ultrasound for obtaining echo signals each used to generate an ultrasonic image will be described based on FIG. 7. The transmit-receive unit 3 performs B-mode image ultrasonic transmission/reception and color Doppler image ultrasonic transmission/reception between one elastic image ultrasonic transmission/reception and another elastic image ultrasonic transmission/reception, which are used to generate one ultrasonic image. Described concretely, the transmit-receive unit 3 performs ultrasonic transmission/reception B1 for a B-mode image and ultrasonic transmission/reception C1 for a color Doppler image between ultrasonic transmission/reception E1 for an elastic image and ultrasonic transmission/reception E2 for an elastic image, which are used to generate an ultrasonic image G1. The transmit-receive unit 3 performs ultrasonic transmission/reception B2 for a B-mode image and ultrasonic transmission/reception C2 for a color Doppler image between the elastic image ultrasonic transmission/reception E2 and elastic image ultrasonic transmission/reception E3, which are used to generate an ultrasonic image G2. Further, the transmit-receive unit 3 performs ultrasonic transmission/reception B3 for a B-mode image and ultrasonic transmission/reception C3 for a color Doppler image between the elastic image ultrasonic transmission/reception E3 and elastic image ultrasonic transmission/reception E4, which are used to generate an ultrasonic image G3.

When each of the elastic image ultrasonic transmission/reception E1, E2, E3, E4, . . . is being carried out, the transmit-receive unit 3 outputs an echo signal to the elastic image processing unit 5. When each of the B-mode image ultrasonic transmission/reception B1, B2, B3, . . . is being carried out, the transmit-receive unit 3 outputs an echo signal to the B-mode image processing unit 4. Further, when each of the color Doppler image ultrasonic transmission/reception C1, C2, C3, . . . is being performed, the transmit-receive unit 3 outputs an echo signal to the color Doppler image processing unit 21. In response to the echo signals outputted from the transmit-receive unit 3, the elastic image processing unit 5 generates elastic image frame data, the B-mode image processing unit 4 generates B-mode image frame data, and the color Doppler image processing unit 21 generates color Doppler image frame data, respectively. The ultrasonic images are formed as described above based on these frame data.

Since the ultrasonic transmission/reception for the B-mode image and the ultrasonic transmission/reception for the color Doppler image are performed between one elastic image ultrasonic transmission/reception and another elastic image ultrasonic transmission/reception both used to generate one ultrasonic image even by the ultrasonic diagnostic apparatus 20 of the present embodiment described above, a frame rate can be enhanced.

In a manner similar to the first embodiment, displacements in position between the elastic image, the B-mode image and the color Doppler image can be suppressed.

Although the invention has been described above by the preferred embodiments, it is needless to say that the preset invention can be changed in various ways within the scope not departing from the gist thereof. For example, an ultrasonic image obtained by combining only two of an elastic image and a color Doppler image together may be generated and displayed. A non-elastic image processing unit is not limited to the B-mode image processing unit 4 and the color Doppler image processing unit 21.

In the second embodiment, the ultrasonic image obtained by combining the B-mode image frame data and the color Doppler image frame data together, and the ultrasonic image obtained by combining the B-mode image frame data and the elastic image frame data together may be displayed on the display unit 7 side by side without displaying the ultrasonic image obtained by combining the B-mode image frame data, the color Doppler image frame data and the elastic image frame data together.

Further, as a physical quantity related to the elasticity of the biological tissue, the distortion and elastic modulus of the biological tissue are known in addition to the displacement due to the deformation of the biological tissue. The distortion or elasticity of each part in the biological tissue may be calculated based on two echo signals from the biological tissue, which are different in time from each other, to generate an elastic image in a manner similar to the above.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
an ultrasonic probe configured to transmit and receive ultrasound on a biological tissue;
a transmit-receive unit configured to:
drive the ultrasonic probe to perform the transmission/reception of the ultrasound and output each echo signal received by the ultrasonic probe;
cause a first elastic image ultrasonic transmission/reception operation and a second elastic image ultrasonic transmission/reception operation to be performed at predetermined time intervals for obtaining echo signals each used to generate an elastic image of the biological tissue; and
cause a non-elastic image ultrasonic transmission/reception operation to be performed for obtaining an echo signal used to generate a non-elastic image related to the biological tissue;
an elastic image processing unit configured to calculate a physical quantity related to elasticity of the biological tissue based on the two echo signals obtained by the first and second elastic image ultrasonic transmission/reception operations and generate the elastic image based on the physical quantity; and
a non-elastic image processing unit configured to generate the non-elastic image based on the echo signal obtained by the non-elastic image ultrasonic transmission/reception operation.

2. The ultrasonic diagnostic apparatus according to claim 1, wherein the transmit-receive unit is configured to:
output the echo signal to the elastic image processing unit after each elastic image ultrasonic transmission/reception operation; and
output the echo signal to the non-elastic image processing unit after the non-elastic image ultrasonic transmission/reception operation.

3. The ultrasonic diagnostic apparatus according to claim 1, wherein the non-elastic image processing unit comprises a B-mode image processing unit configured to generate a B-mode image, and
wherein the transmit-receive unit is configured to cause a B-mode image ultrasonic transmission/reception operation to be performed for obtaining an echo signal used to generate the B-mode image as the non-elastic image ultrasonic transmission/reception operation.

4. The ultrasonic diagnostic apparatus according to claim 3, wherein the transmit-receive unit is configured to:
output the echo signal to the elastic image processing unit after each elastic image ultrasonic transmission/reception operation; and
output the echo signal to the B-mode image processing unit after the B-mode image ultrasonic transmission/reception operation.

5. The ultrasonic diagnostic apparatus according to claim 3, further comprising a combination unit configured to combine the elastic image and the B-mode image.

6. The ultrasonic diagnostic apparatus according to claim 1, wherein the non-elastic image processing unit comprises a color Doppler image processing unit configured to generate a color Doppler image, and
wherein the transmit-receive unit is configured to cause a color Doppler image ultrasonic transmission/reception operation to be performed for obtaining an echo signal used to generate the color Doppler image as the non-elastic image ultrasonic transmission/reception operation.

7. The ultrasonic diagnostic apparatus according to claim 6, wherein the transmit-receive unit is configured to:
output the echo signal to the elastic image processing after elastic image ultrasonic transmission/reception operation;
output the echo signal to the color Doppler image processing unit after the color Doppler image ultrasonic transmission/reception operation.

8. The ultrasonic diagnostic apparatus according to claim 6, wherein the color Doppler image processing unit comprises an extraction part configured to extract a signal indicative of a blood flow component from the echo signal.

9. The ultrasonic diagnostic apparatus according to claim 6, further comprising a combination unit configured to combine the elastic image and the color Doppler image.

10. The ultrasonic diagnostic apparatus according to claim 8, further comprising a combination unit configured to combine the elastic image and the color Doppler image.

11. The ultrasonic diagnostic apparatus according to claim 1, wherein the non-elastic image processing unit comprises a plurality of non-elastic image processing units, and wherein the non-elastic image processing units are configured to generate non-elastic images different in type from one another, and
wherein the transmit-receive unit configured to cause transmission/reception for the respective non-elastic images to be performed as the non-elastic image ultrasonic transmission/reception.

12. The ultrasonic diagnostic apparatus according to claim 11, wherein upon the transmit-receive unit is configured to:
output the echo signal to the elastic image processing unit after each elastic image ultrasonic transmission/reception operation; and
output the echo signal to its corresponding non-elastic image processing unit of the non-elastic image processing units after the corresponding non-elastic image ultrasonic transmission/reception operation.

13. The ultrasonic diagnostic apparatus according to claim 11, wherein each of the non-elastic image processing units comprises a B-mode image processing unit configured to generate a B-mode image and a color Doppler image processing unit for generating a color Doppler image, and
wherein the transmit-receive unit is configured to cause a B-mode image ultrasonic transmission/reception operation to be performed for obtaining an echo signal used to generate the B-mode image and a color Doppler image ultrasonic transmission/reception operation to be performed for obtaining an echo signal used to generate the color Doppler image as the non-elastic image ultrasonic transmission/reception.

14. The ultrasonic diagnostic apparatus according to claim 13, wherein the transmit-receive unit is configured to:
output the echo signal to the elastic image processing unit after the elastic image ultrasonic transmission/reception operation;
output the echo signal to the B-mode image processing unit after the B-mode image ultrasonic transmission/reception operation; and
output the echo signal to the color Doppler image processing unit after the color Doppler image ultrasonic transmission/reception operation.

15. The ultrasonic diagnostic apparatus according to claim 13, further comprising a combination unit configured to combine the B-mode image and the color Doppler image.

16. A program for causing a computer of an ultrasonic diagnostic apparatus to execute the following:
a transmitting/receiving function that causes a processor to:
drive an ultrasonic probe to cause transmission/reception of ultrasound to be performed on a biological tissue;
output echo signals received by the ultrasonic probe;
cause a first elastic image ultrasonic transmission/reception operation and a second elastic image ultrasonic transmission/reception operation to be performed at predetermined time intervals for obtaining echo signals each used to generate an elastic image of the biological tissue;
cause a non-elastic image ultrasonic transmission/reception operation to be performed between the first and second elastic image ultrasonic transmission/reception operations for obtaining an echo signal used to generate a non-elastic image related to the biological tissue;
an elastic image generating function that causes a processor to calculate a physical quantity related to elasticity of the biological tissue based on the two echo signals obtained by the first and second elastic image ultrasonic transmission/reception operations and generate the elastic image based on the physical quantity; and
a non-elastic image generating function that causes a processor to generate the non-elastic image based on the echo signal obtained by the non-elastic image ultrasonic transmission/reception operation.

17. An ultrasonic diagnosis method comprising:
transmitting and receiving ultrasound on a biological tissue;
performing a first elastic image ultrasonic transmission/reception operation and a second elastic image ultrasonic transmission/reception operation at predetermined time intervals for obtaining echo signals each used to generate an elastic image of the biological tissue; and
performing a non-elastic image ultrasonic transmission/reception operation for obtaining an echo signal used to generate a non-elastic image related to the biological tissue;
calculating a physical quantity related to elasticity of the biological tissue based on the two echo signals obtained by the first and second elastic image ultrasonic transmission/reception operations;
generating the elastic image based on the physical quantity; and
generating the non-elastic image based on the echo signal obtained by the non-elastic image ultrasonic transmission/reception operation.

18. The ultrasonic diagnosis method according to claim 17, further comprising combining the elastic image and the non-elastic image.

19. The ultrasonic diagnosis method according to claim 17, wherein the non-elastic image is a B-mode image, the method further comprising combining the elastic image and the B-mode image.

20. The ultrasonic diagnosis method according to claim 17, wherein the non-elastic image is a color Doppler image, the method further comprising combining the elastic image and the color Doppler image.
